(19) United States
(12) Patent Application Publication
(10) Pub. No.: US 2004/0038864 A1
(43) Pub. Date: Feb. 26, 2004

(54) Use of dimethyl sulfone as isotonicity agent

(76) Inventors: Per Balschmidt, Espergaerde (DK); Svend Havelund, Bagsvaerd (DK)

Correspondence Address: Reza Green, Esq., Novo Nordisk Pharmaceuticals, Inc., 100 College Road West, Princeton, NJ, 08540, US

(21) Appl. No.: 10/602,456

(22) Filed: Jun. 23, 2003

Related U.S. Application Data
(60) Provisional application No. 60/394,154, filed on Jul. 3, 2002.

(30) Foreign Application Priority Data
Jun. 27, 2002 (DK) PA 2002 01007

(51) Int. Cl. A61K 38/00

(52) U.S. Cl. 514/2

(57) ABSTRACT

A pharmaceutical composition for parenteral administration comprising a peptide and dimethyl sulfone and use of dimethyl sulfone as an isotonicity agent in a pharmaceutical composition for parenteral administration, especially a pharmaceutical composition for parenteral administration comprising a peptide as the active ingredient.

### FIELD OF THE INVENTION

The invention relates to a pharmaceutical composition for parenteral administration comprising a peptide and dimethyl sulfone and to the use of dimethyl sulfone as isotonicity agent in a pharmaceutical composition, especially a pharmaceutical composition comprising a peptide as the active ingredient.

### BACKGROUND OF THE INVENTION

To avoid pain or tissue damage, pharmaceutical compositions for parenteral administration comprise an isotonicity agent to provide tonicity or osmolarity close to the body fluids at the administration site. Conventional isotonicity agents for parenteral peptide compositions are glycerol, dextrose, mannitol, lactose and salts such as sodium chloride.

The choice of isotonicity agent will affect the properties of the preparation as revealed by the chemical stability. For example aldehyde impurities of glycerol may result in transformation of the peptide thus resulting in a product with deteriorated stability.

Dimethyl sulfone is a well known organic compound which has been disclosed for different applications.

U.S. Pat. Nos. 4,863,748 and 4,616,039 disclose dimethyl sulfone for use as a dietary supplement. U.S. Pat. No. 4,973,605, U.S. Pat. No. 4,559,329 and U.S. Pat. No. 4,514,421 disclose dietary and pharmaceutical uses of dimethyl sulfone. U.S. Pat. No. 4,568,547 discloses the use of dimethyl sulfone as a tabletting and granulating aid for pharmaceutically active agents. U.S. Pat. No. 4,296,130 and US 4,477,469 disclose preparations containing dimethyl sulfone for softening skin and nails or for diluting blood. CA 1988-568512 discloses a formulation for treating cancer comprising dimethyl sulfone for enhancing or altering the penetration of the active agent to the tumour. WO 01/26642 discloses a method for treating neurobehavioral disorders by administering a composition comprising amino acids, neurotransmitter precursors, vitamins, inhibitors of neurotransmitter degradation and/or immune function enhancers. Among the vitamins listed is dimethyl sulfone.

The present invention is based on the finding that dimethyl sulfone is useful as an isotonicity agent in pharmaceutical compositions for parenteral administration, especially pharmaceutical compositions for parenteral administration which comprise a peptide as the active ingredient.

### DEFINITIONS

The following is a detailed definition of the terms used in the specification:

The term “peptide” as used herein is intended to include a compound formed by linking at least two amino acids through a peptide bond. The term comprises oligopeptides containing fewer than 10 amino acids as well as polypeptides containing at least 10 amino acids. The term includes naturally occurring peptides, including proteins, as well as synthetic peptides. The term is also intended to include modified peptides, eg alkylated, acylated peptides, etc.

The term “parenteral administration” as used herein means that the administration is not through the alimentary canal but rather through some other route, such as via the subcutaneous, intramuscular, intrathecal, pulmonal, intravenous, intradermal, intraspinal or intrasternal route. The term also covers ophthalmic administration and topical administration.

The term “analogue” as used herein designates a peptide wherein one or more amino acid residues of the parent peptide have been substituted by another amino acid residue and/or wherein one or more amino acid residues of the parent peptide have been deleted and/or wherein one or more amino acid residues have been added to the parent peptide. Such addition can take place either in the peptide, at the N-terminal end or at the C-terminal end of the parent peptide, or any combination thereof.

The term “derivative” as used herein designates a peptide in which one or more of the amino acid residues of the parent peptide or analogue of the parent peptide have been chemically modified, eg by alkylation, acylation, ester formation or amide formation.

### DESCRIPTION OF THE INVENTION

The present invention relates to a pharmaceutical composition for parenteral administration, which comprises a peptide and dimethyl sulfone.

Dimethyl sulfone which is also designated methylsulfonylmethane or MSM may be prepared from dimethyl sulfoxide by oxidation. It can be obtained in highly purified form as crystals with a melting point at about 110° C. and has a well defined boiling point at 238° C. It is commercially available at a low price. It is non-toxic and non-allergenic. Furthermore, it is very soluble in water. These properties make it very attractive for use in pharmaceutical compositions. Dimethyl sulfone is very stable and does not deteriorate the peptide in the pharmaceutical composition. Accordingly, very stable pharmaceutical compositions are provided.

Dimethyl sulfone is used in an amount to make the pharmaceutical composition isotonic with the site of administration. The amount has to be adjusted based on th other ingredients of the pharmaceutical composition which may also contribute to isotonicity.

In one embodiment, the amount of dimethyl sulfone is of from 40 to 400 mM, such as of from 125 to 350 mM.

In another embodiment, the pharmaceutical composition is a solution.

In yet another embodiment, the pharmaceutical composition is a suspension.

The pharmaceutical composition is intended for parenteral administration. In one embodiment, the pharmaceutical composition is adapted for administration by injection or infusion, such as subcutaneous administration, intramuscular administration or intravenous administration.

In another embodiment, the pharmaceutical composition is adapted for pulmonal administration.

In yet her embodiment, the pharmaceutical composition is adapted for ophthalmic administration or topical administration.

The pharmaceutical composition comprises a peptide as active ingredient. In one embodiment, the peptide is human growth hormone, GLP-1, GLP-2, insulin, Factor VII, Factor VIII, erythropoeitin (EPO), glucagon, interleukin, such as interleukin-2 (IL-2), interferon-α or interferon-β, or an analogue thereof, or a derivative of any such peptide or analogue.

In another embodiment, the peptide is human insulin or an analogue thereof, or a derivative of human insulin or the human insulin analogue, such as human insulin, Asp(B28)-human insulin, Lys(B28) Pro(B29)-human insulin, Lys(B3) Glu(B29)-human insulin, N^{εB29}-tetradecanoyl des (B30)-human insulin, Gly(A21) Arg(B31) Arg(B32)-human insulin or N^{εB29}-litocholoyl-γ-glutamyl des (B30)-human insulin.

In yet another embodiment, the peptide is Gly(8)-human GLP-1, Arg(34), N-ε-(γ-Glu(N-α-hexadecanoyl))-Lys(26)-human GLP-1(7-37)OH or Gly(2)-human GLP-2.

In a further aspect the invention relates to the use of dimethyl sulfone as an isotonicity agent in a pharmaceutical composition for parenteral administration.

In yet a further aspect, the invention relates to the use of dimethyl sulfone as an isotonicity agent in a pharmaceutical composition for parenteral administration comprising a peptide as the active ingredient.

In one embodiment, the amount of dimethyl sulfone in the pharmaceutical composition is of from 40 to 400 mM, such as of from 125 to 350 mM.

In another embodiment, the pharmaceutical composition is a solution.

In yet another embodiment, the pharmaceutical composition is a suspension.

In still another embodiment, the pharmaceutical composition is adapted for administration by injection or infusion, such as subcutaneous administration, intramuscular administration or intravenous administration.

In a further embodiment, the pharmaceutical composition is adapted for pulmonal administration.

In a further embodiment, the pharmaceutical composition is adapted for ophthalmic administration or topical administration.

In yet a further embodiment, the peptide is human growth hormone, GLP-1, GLP-2, insulin, Factor VII, Factor VIII, erythropoeitin (EPO), glucagon, interleukin, such as interleukin-2 (IL-2), interferon-α or interferon-β, or an analogue thereof, or a derivative of any such peptide or analogue.

In still a further embodiment, the peptide is human insulin or an analogue thereof, or a derivative of human insulin or the human insulin analogue, such as human insulin, Asp(B28)-human insulin, Lys(B28) Pro(B29)-human insulin, Lys(B3) Glu(B29)-human insulin, N^{εB29}-tetradecanoyl des (B30)-human insulin, Gly(A21) Arg(B31) Arg(B32)-human insulin or N^{εB29}-litocholoyl-γ-glutamyl des (B30)-human Insulin.

In another embodiment, the peptide is Gly(8)-human GLP-1, Arg(34), N-ε-(γ-Glu(N-α-hexadecanoyl))-Lys(26)-human GLP-1(7-37)OH or Gly(2)-human GLP-2.

Pharmaceutical Compositions

The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions according to the invention are intended for parenteral administration, such as subcutaneous, intramuscular, intrathecal, intravenous, intradermal, intraspinal or intrasternal administration. Other suitable administration routes are ophthalmic administration or topical administration for treating open wounds, ulcers, bed-sores, pressure sores and burns.

It will be appreciated that the preferred route of administration will depend on the general condition and age of the subject to be treated, the nature of the condition to be treated and the active ingredient chosen.

Suitable administration forms include sterile aqueous and non-aqueous injectable solutions, dispersions, suspensions or mulsions as well as sterile powders to be reconstituted in sterile injectable solutions or dispersions prior to use. Depot injectable formulations are also contemplated as being within the scope of the present invention.

Other suitable administration forms include ophthalmic preparations such as eye drops and eye ointments and topical preparations such as wound dressings.

### EXAMPLES

### Example 1

Formulation of Dissolved Human Insulin Preparation Containing Dimethyl Sulfone

74.9 mg of Zn-crystallized human insulin is dispersed in 2 ml of water and dissolved by addition of 32.5 μl of 2N hydrochloric acid. Then the following ingredients are added:

2.0 ml of 160 mM m-cresol solution

2.0 ml of 160 mM phenol solution

376 mg of dimethyl sulfone

1.0 ml of 140 mM disodium hydrogen phosphate solution

1.0 ml of 200 mM sodium chloride solution

10 ml of water

The pH is adjusted to 7.3 with diluted hydrochloric acid or sodium hydroxide and water is added to a total volume of 20.0 ml. The resulting solution is finally sterilized by filtration.

### Example 2

Comparison of the Chemical Stability of 3 Formulations of Dissolved Human Insulin

Formulation I is prepared according to example 1.

Formulation II is prepared according to example 1 with the exception that the dimethyl sulfone is omitted.

Formulation III is prepared according to example 1 with the exception that the dimethyl sulfone is replaced by 368 mg of glycerol.

The formulations were stored in closed 1 ml HPLC vials at 4° C. and at 25° C., respectively. After storage for 10 weeks the formulations were analyzed by reverse phase HPLC on a 4.6 mm×150 mm Waters SymmetryShield RP₈ (3.5 μm) column eluted at 30° C. by a buffer system A (0.2 M sodium sulphate, 0.04 M sodium phosphate, pH 7.2 in 10% (v/v) acetonitrile) with 19% B (70% (v/v) acetonitrile ) for 21 min, then with 24% B for 30 min and finally with a linear gradient from 24% B to 39% B over 30 min.

The AUC for the side peaks in percentage of the total AUC for the insulin-relat d peaks was calculated as a measure of purity. Thus, a low number indicates a high degree of purity. The difference in purity between the human insulin solutions stored at 4° C. and 25° C., respectively, is shown in the table for each of the formulations.

| Formulation | Δ % Purity |
|---|---|
| I (dimethyl sulfone) | 1.64 |
| II (no isotonicity agent) | 1.62 |
| III (glycerol) | 1.92 |

The results show that the content of dimethyl sulfone does not impair the chemical stability of the insulin in the formulation and that the insulin is substantially less stable when dimethyl sulfone is replaced by glycerol in the formulation.

### Example 3

Formulation of NPH-Crystallized Human Insulin Preparation Containing Dimethyl Sulfone

Solution A:

74.5 mg of Zn-crystallized human insulin is dispersed in 2 ml of water and dissolved by addition of 34 μl of 2N hydrochloric acid. Then the following ingredients are added:

650 μl of 160 mM m-cresol solution

430 μl of 160 mM phenol solution

176 mg of dimethyl sulfone

35.8 μl of zinc chloride solution (10 mg Zn/ml)

750 μl of protamine sulphate solution (10 mg/ml)

Water is added to a total volume of 10 ml.

Solution B:

The following ingredients are mixed:

2.0 ml of 140 mM disodium hydrogen phosphate

650 μl of 160 mM m-cresol

430 μl of 160 mM phenol

176 mg of dimethyl sulfone

8 μl of 2N sodium hydroxide

Water is added to a total volume of 10 ml. Solution A and solution B are sterilized by filtration and then mixed. The resulting suspension is left at 23° C. and after overnight standing the crystallization is complete.

### Example 4

Formulation of a Preparation of Dissolved Asp(B28)-Human Insulin Analogue Containing Dimethyl Sulfone

151.9 mg of Zn-free Asp(B28)-human insulin (can be prepared as described in eg EP 214 826) is dispersed in 2 ml of water and dissolved by addition of 65 μl of 2N hydrochloric acid. Then the following excipients are added with gentle stirring:

78.4 μl of zinc chloride solution (10 mg Zn/ml)

4.0 ml of 160 mM m-cresol solution

4.0 ml of 160 mM phenol solution

752 mg of dimethyl sulfone

50 mg of disodium hydrogen phosphate, dihydrate

23.5 mg of sodium chloride

25 ml of water

The pH is adjusted to 7.3 with diluted hydrochloric acid or sodium hydroxide and water is added to a total volume of 40.0 ml. The resulting solution is finally sterilized by filtration.

### Example 5

Formulation of a Dissolved Preparation of Arg(34), N-ε-(γ-Glu(N-α-hexadecanoyl))-Lys(26)-Human GLP-1(7-37)OH Containing Dimethyl Sulfone

The following ingredients are mixed:

3.2 ml of 50 mM disodium hydrogen phosphate solution

10.6 ml of 100 mM phenol solution

382 mg of dimethyl sulfone

5 ml of water

The pH is adjusted to 7.4 with diluted hydrochloric acid and water is added to a total volume of 20 ml.

40 mg of Arg(34), N-ε-(γ-Glu(N-α-hexadecanoyl))-Lys(26)-human GLP-1(7-37)OH (can be prepared as described in eg WO 98/08871) is added and dissolved by gentle stirring. The resulting solution is sterilized by filtration, if necessary, after readjustment of the pH to 7.4.

### Example 6

Formulation of a Dissolved Preparation of Human Growth Hormone Containing Dimethyl Sulfone

The following ingredients are mixed:

6.8 mg of L-histidine

30 mg of poloxamer 188

207 mg of dimethyl sulfone

3.2 ml of 100 mM phenol solution

5 ml of water

The pH is adjusted to 6.2 with diluted hydrochloric acid and water is added to a total volume of 10 ml.

67 mg of human growth hormone (can be prepared as described in eg EP 217 814 or EP 218 651) is added and dissolved by gentle stirring. The resulting solution is sterilized by filtration, if necessary, after readjustment of the pH to 6.2.

1. A pharmaceutical composition for parenteral administration, which comprises a peptide and dimethyl sulfone.

2. A pharmaceutical composition according to claim 1, wherein the amount of dimethyl sulfone is of from 40 to 400 mM.

3. A pharmaceutical composition according to claim 2, wherein amount of dimethyl sulfone is of from 125 to 350 mM.

4. A pharmaceutical composition according to any one of the claims 1 to 3, wherein the composition is a solution.

5. A pharmaceutical composition according to any one of the claims 1 to 3, wherein the composition is a suspension.

6. A pharmaceutical composition according to any one of the preceding claims, which is suitable for administration by injection or infusion.

7. A pharmaceutical composition according to claim 6, which is suitable for subcutaneous administration.

8. A pharmaceutical composition according to claim 6, which is suitable for intramuscular administration.

9. A pharmaceutical composition according to claim 6, which is suitable for intravenous administration.

10. A pharmaceutical composition according to any one of the preceding claims 1 to 5, which is suitable for pulmonal administration.

11. A pharmaceutical composition according to any one of the preceding claims 1 to 5, which is suitable for ophthalmic administration or topical administration.

12. A pharmaceutical composition according to any one of the preceding claims, wherein the peptide is human growth hormone, GLP-1, GLP-2, insulin, Factor VII, Factor VIII, erythropoeitin (EPO), glucagon, interleukin, such as interleukin-2 (IL-2), interferon-α or interferon-β, or an analogue thereof, or a derivative of any such peptide or analogue.

13. A pharmaceutical composition according to claim 12, wherein the peptide is human insulin or an analogue thereof, or a derivative of human insulin or the human insulin analogue.

14. A pharmaceutical composition according to claim 13, wherein the peptide is human insulin.

15. A pharmaceutical composition according to claim 13, wherein the peptide is Asp(B28)-human insulin.

16. A pharmaceutical composition according to claim 13, wherein the peptide is Lys(B28) Pro(B29)-human insulin.

17. A pharmaceutical composition according to claim 13, wherein the peptide is Lys(B3) Glu(B29)-human insulin.

18. A pharmaceutical composition according to claim 13, wherein the peptide is N^{εB29}-tetradecanoyl des (B30)-human insulin.

19. A pharmaceutical composition according to claim 13, wherein the peptide is Gly(A21) Arg(B31) Arg(B32)-human insulin.

20. A pharmaceutical composition according to claim 13, wherein the peptide is N^{εB29}-litocholoyl-γ-glutamyl des (B30)-human insulin.

21. A pharmaceutical composition according to claim 12, wherein the peptide is Gly(8)-human GLP-1.

22. A pharmaceutical composition according to claim 12, wherein the peptide is Arg(34), N-ε-(γ-Glu(N-α-hexadecanoyl))-Lys(26)-human GLP-1(7-37)0H.

23. A pharmaceutical composition according to claim 12, wherein the peptide is Gly(2)-human GLP-2.

24. Use of dimethyl sulfone as an isotonicity agent in a pharmaceutical composition for parenteral administration.

25. Use of dimethyl sulfone as an isotonicity agent in a pharmaceutical composition for parenteral administration comprising a peptide.

26. Use according to claims 24 or 25, wherein the amount of dimethyl sulfone in the pharmaceutical composition is of from 40 to 400 mM.

27. Use according to claim 26, wherein the amount of dimethyl sulfone in the pharmaceutical composition is of from 125 to 350 mM.

28. Use according to any one of the claims 24 to 27, wherein the composition is a solution.

29. Use according to any one of the claims 24 to 27, wherein the composition is a suspension.

30. Use according to any one of the claims 24 to 29, wherein the composition is suitable for administration by injection or infusion.

31. Use according to claim 30, wherein the composition is suitable for subcutaneous administration.

32. Use according to claim 30, wherein the composition is suitable for intramuscular administration.

33. Use according to claim 30, wherein the composition is suitable for intravenous administration.

34. Use according to any one of the claims 24 to 29, wherein the composition is suitable for pulmonal administration.

35. Use according to any one of the claims 24 to 29, wherein the composition is suitable for ophthalmic administration or topical administration.

36. Use according to any one of the preceding claims 24 to 35, wherein the peptide is human growth hormone, GLP-1, GLP-2, insulin, Factor VII, Factor VIII, erythropoeitin (EPO), glucagon, interleukin, such as interleukin-2 (IL-2), interferon-α or interferon-β, or an analogue thereof, or a derivative of any such peptide or analogue.

37. Use according to claim 36, wherein the peptide is human insulin or an analogue thereof, or a derivative of human insulin or the human insulin analogue.

38. Use according to claim 37, wherein the peptide is human insulin.

39. Use according to claim 37, wherein the peptide is Asp(B28)-human insulin.

40. Use according to claim 37, wherein the peptide is Lys(B28) Pro(B29)-human insulin.

41. Use according to claim 37, wherein the peptide is Lys(B3) Glu(B29)-human insulin.

42. Use according to claim 37, wherein the peptide is N^{εB29}-tetradecanoyl des (B30)-human insulin.

43. Use according to claim 37, wherein the peptide is Gly(A21) Arg(831) Arg(B32)-human insulin.

44. Use according to claim 37, wherein the peptide is N^{εB29}-litocholoyl-γ-glutamyl des (B30)-human insulin.

45. Use according to claim 36, wherein the peptide is Gly(8)-human GLP-1.

46. Use according to claim 36, wherein the peptide is Arg(34), N-ε-(γ-Glu(N-α-hexadecanoyl))-Lys(26)-human GLP-1(7-37)OH.

47. Use according to claim 36, wherein the peptide is Gly(2)-human GLP-2.
